SEQUENCE LISTING (28 sequences)

SEQ ID NO: 1 - protein, 8 aa, Acinetobacter sp.
RFSAWGAE

SEQ ID NO: 2 - protein, 9 aa, Acinetobacter sp.
ISRFAWGEV

SEQ ID NO: 3 - protein, 6 aa, Bovine
NEALEK

SEQ ID NO: 4 - protein, 7 aa, Bovine
LKKVHEE

SEQ ID NO: 5 - protein, 7 aa, Bovine
EALEKQL

SEQ ID NO: 6 - protein, 7 aa, Bovine
ELEDKQN

SEQ ID NO: 7 - protein, 6 aa, Bovine
KKVHEE

SEQ ID NO: 8 - protein, 6 aa, Bovine
EIRDLR

SEQ ID NO: 9 - protein, 8 aa, Bovine
EQEIRDLR

SEQ ID NO: 10 - protein, 5 aa, Bovine/Acinetobacter sp.
RPVDQ

SEQ ID NO: 11 - protein, 14 aa, Acinetobacter sp.
AIGSRPVDQHLKAL

SEQ ID NO: 12 - protein, 14 aa, Bovine
QVYYRPVDQYSNQN

SEQ ID NO: 13 - protein, 6 aa, Bovine
RFAWGE

SEQ ID NO: 14 - protein, 7 aa, Bovine
RFSWGAE

SEQ ID NO: 15 - protein, 6 aa, Bovine
  MISC_FEATURE (3)..(3): any amino acid
RFXWXE

SEQ ID NO: 16 - protein, 7 aa, Bovine
  MISC_FEATURE (3)..(3): any amino acid
RFXWXXE

SEQ ID NO: 17 - protein, 15 aa, Bovine
QNFISRFAWGEVNSR

SEQ ID NO: 18 - protein, 15 aa, Bovine
RGSLSRFSWGAEGQK

SEQ ID NO: 19 - protein, 6 aa, Bovine
KEALEK

SEQ ID NO: 20 - protein, 7 aa, Bovine
IEKVEEE

SEQ ID NO: 21 - protein, 7 aa, Bovine
EALEYGL

SEQ ID NO: 22 - protein, 7 aa, Bovine
ALEDKSN

SEQ ID NO: 23 - protein, 7 aa, Bovine
EAYAKQL

SEQ ID NO: 24 - protein, 6 aa, Bovine
KKVKEE

SEQ ID NO: 25 - protein, 6 aa, Bovine
EIRDLE

SEQ ID NO: 26 - protein, 8 aa, Bovine
EQIVRDAR

SEQ ID NO: 27 - protein, 14 aa, Bovine
RALIALDKSNFIEA

SEQ ID NO: 28 - protein, 15 aa, Bovine
KQLQELEDKQNADIS

(19) United States
(12) Patent Application Publication
(10) Pub. No.: US 2005/0244895 A1
(43) Pub. Date: Nov. 3, 2005

(54) Diagnosis of demyelinating or spongiform disease

(76) Inventors: Alan Ebringer, London (GB); Clyde Donald Dycee Wilson, London (GB)

Correspondence Address: Joseph T Leone;Dewitt Ross & Stevens, Excelsior Financial Centre, Suite 401 8000 Excelsior Drive, Madison, WI, 53717-1914, US

(21) Appl. No.: 10/494,781

(22) PCT Filed: Nov. 8, 2002
(86) PCT No.: PCT/GB02/05056

§ 371 Date: May 2, 2005

(30) Foreign Application Priority Data
Nov. 9, 2001 (GB) 0127000.8
Feb. 4, 2002 (GB) 0202562.5

(51) Int. Cl. G01N 33/53; G01N 33/567; G01N 33/554; G01N 33/569

(52) U.S. Cl. 435/7.2; 435/7.32

(57) ABSTRACT

A method for detecting demyelinating disease or spongiform encephalopathy in verterbrates comprising testing a biological sample obtained from the vertebrate for antibodies capable of binding to both Acinetobacter antigens and to prion antigens. This test can be combined with previously described tests involving measurement of antibodies capable of binding to myelin and/or neurofilament and or Acinetobacter antigens. A kit for performing the test is also described.

This invention relates to the diagnosis of de-myelinating diseases and spongiform encephalopathies in animals and humans.

In our published application WO 98/13694 we have disclosed a new diagnostic test for spongiform encephalopathies and other de-myelinating conditions in mammals. The test disclosed in our prior application is based on a model of the genesis of this pathological state which is applicable to the various forms in which it is manifest in humans and animals. In relation to the bovine spongiform disease this model provides an alternative to the current theory based on the formation of prions. Briefly, this new model is based on the phenomenon of molecular mimicry according to which mammals exposed to certain bacteria having peptide sequences which mimic myelin peptides experience an auto-immune reaction. In our prior application we indicated that human de-myelinating diseases were also open to the same explanation according to our new model disclosed therein.

In our subsequent published application WO 99/47932 we confirmed the presence of elevated levels of certain antibodies in human sera of patients suffering from multiple sclerosis (MS). These are the IgA antibodies to Acinetobacter species e.g. Acinetobacter calcoaceticus, the same organisms for which antibodies were previously found in BSE sera. Similar results have been obtained for Creutzfeldt-Jakob disease (CJD). Tests for antibodies in sera from patients who had died of CJD also show increased levels, this being especially marked for the IgA antibody sub-class. The same IgA specificity also applies to bovine sera used for the tests described in our above-mentioned copending application.

As indicated in our earlier applications, Acinetobacter calcoaceticus is one species of Acinetobacter which provides an antigen which stimulates the formation of antibodies which cross-react with the mammalian myelin. Antibodies have been demonstrated to react with several species of Acinetobacter including 17905, AC606, SP13TV, 105/85, and 11171. Exemplary data are tabulated below.

TABLE
Antibody responses (Mean +/− S.E.) in different strains of Acinetobacter.

| | | BSE | | t- | Statistical |
|---|---|---|---|---|---|
| Number | Name | Positive | Controls | value | Significance |
| 1 | A. calcoaceticus | 0.668 +/− | 0.298 +/− | 8.66 | p < 0.001 |
| | (sp1) | 0.031 | 0.098 | | |
| 2 | A. baumanni | 0.452 +/− | 0.251 +/− | 7.02 | p < 0.001 |
| | (sp2) | 0.013 | 0.030 | | |
| 3 | Acinetobacter | 0.402 +/− | 0.230 +/− | 9.27 | p < 0.001 |
| | (sp3) | 0.011 | 0.015 | | |
| 4 | A. haemolyticus | 0.376 +/− | 0.237 +/− | 7.79 | p < 0.001 |
| | (sp4) | 0.012 | 0.013 | | |
| 5 | A. junii | 0.245 +/− | 0.145 +/− | 5.95 | p < 0.001 |
| | (sp5) | 0.011 | 0.011 | | |
| 6 | Acinetobacter | 0.399 +/− | 0.222 +/− | 6.74 | p < 0.001 |
| | (sp6) | 0.016 | 0.021 | | |
| 7 | A. johnsonii | 0.627 +/− | 0.340 +/− | 13.52 | p < 0.001 |
| | (sp7) | 0.014 | 0.014 | | |
| 8 | A. lwoffii | 0.494 +/− | 0.228 +/− | 8.07 | p < 0.001 |
| | (sp8) | 0.024 | 0.016 | | |
| 9 | Acinetobacter | 0.506 +/− | 0.268 +/− | 8.63 | p < 0.001 |
| | (sp9) | 0.016 | 0.023 | | |
| 10 | Acinetobacter | 0.383 +/− | 0.266 +/− | 6.34 | p < 0.001 |
| | (sp10) | 0.010 | 0.017 | | |
| 11 | Acinetobacter | 0.425 +/− | 0.254 +/− | 6.65 | p < 0.001 |
| | (sp16) | 0.015 | 0.022 | | |
| 12 | Acinetobacter | 0.415 +/− | 0.223 +/− | 5.94 | p < 0.001 |
| | (sp17) | 0.020 | 0.026 | | |

In carrying out the necessary test for antibodies which bind to an epitope present in or derived from the Acinetobacter species, the antigen used in the test may be the whole organism or at least one prepared peptide sequence corresponding to an Acinetobacter epitope. Alternatively, peptide sequences may be used which have minor variations in amino-acid sequence from the above-mentioned epitopes or prepared peptides but are conformationally sufficiently similar to them that they also bind to the relevant antibodies. For example, peptides having the sequence RFSAWGAE (SEQ ID NO: 1) or ISRFAWGEV (SEQ ID NO: 2) may be used.

In our third published application WO 00/31545 we have disclosed a further variation on the two previous applications in which antibodies which bind to myelin or neurofilaments are tested for by the use of test antigens derived from or based on sequences present in these materials. Examples of peptide antigens useful for such purpose are NEALEK (SEQ ID NO: 3), LKKVHEE (SEQ ID NO: 4), EALEKQL (SEQ ID NO: 5), ELEDKQN (SEQ ID NO: 6), KKVHEE (SEQ ID NO: 7), EIRDLR (SEQ ID NO: 8), and EQEIRDLR (SEQ ID NO: 9). In this third application we also disclosed a method of combining the measurement of antibodies capable of binding to Acinetobacter species with the measurement of antibodies capable of binding to myelin and/or neurofilaments, or antigenic parts of these. This measurement was described as the MAN index (short for Myelin Acinetobacter Neurofilaments).

The entire contents of the three earlier applications identified above, and any parts thereof, are to be read as incorporated by reference into the present disclosure and should be referred to if and where necessary to provide a full understanding and complete description of the present invention.

We have now discovered a relationship between certain Acinetobacter epitopes and sequences present in the prion molecule. One example of such a relationship is a sequence similarity between Acinetobacter-UDP-N-acetylglucosamnine 1-carboxy-vinyl transferase and the bovine prion molecule. This similarity involves the identity of the sequence RPVDQ (SEQ ID NO: 10), which occurs between positions 121 and 125 of the Acinetobacter sequence:—
AIGSRPVDOHLKAL (SEQ ID NO: 11)
   and positions 175 and 179 of the bovine prion molecule:—
QVYYRPVDOYSNQN (SEQ ID NO: 12)

It will thus be appreciated that cattle affected by exposure to Acinetobacter species will have antibodies that bind to this common sequence. This linkage between an Acinetobacter sequence and a prion sequence therefore provides the possibility of alternative or additional test antigens for testing sera of mammals including cattle, sheep and humans for the presence of the de-myelinating or spongiform diseases mentioned above. Spongiform encephalopathies and de-myelinating diseases may include BSE in cattle, Scrapie in sheep and CJD and/or multiple sclerosis (MS) in humans.

Such a test antigen may comprise the specified common sequence or a larger peptide containing the common sequence or a related sequence e.g. a closely homologous and cross-reactive sequence which may contain modified or additional amino acid residues totalling at least 15 residues.

In the accompanying drawings, FIG. 1 shows the 3-dimensional structures of the corresponding parts of the Acinetobacter and prion molecules in which the Aspartic acid and Arginine residues are of especial significance. FIG. 2 shows the cross-reacting epitope in the prion molecule.

According to the present invention, a method for detecting a de-myelinating disease or spongiform encephalopathy in vertebrates comprises testing a biological sample obtained from the vertebrate for antibodies of any isotype capable of binding to antigens present in Acinetobacter or part thereof and also capable of binding to antigens present in prions (including normial or denatured prions) of the same vertebrate origin. The present invention also comprises a method for detecting a de-myelinating disease or spongiform encephalopathy in vertebrates which comprises testing a biological sample obtained from the vertebrate for antibodies of any isotype capable of binding to an antigen which contains the peptide sequence RPVDQ (SEQ ID NO: 10) or a related sequence as indicated above. Such antigens may include use of a peptide having the sequence AIGSRPVDOHLKAL (SEQ ID NO: 11) or a peptide having the sequence QVYYRPVDOYSNQN (SEQ ID NO: 12) or a related sequence as indicated above.

The present invention also comprises a method of combining of the measurement of antibodies capable of binding to prions with the measurement of antibodies capable of binding to myelin and/or neurofilament and/or Acinetobacter species, or antigenic parts of these.

This measurement is therefore an extension of the MAN index referred to above, in which measurements are taken of the level of antibodies to prions and combined with measurements of any one or more of the above antibodies (i.e. antibodies to Myelin, Acinetobacter, and Neurofilaments, or antigenic parts of any of these) by multiplication to produce a figure for the revised MAN index (which can be described in its simplest forms as the MPN index or MAPN index). One molecule present in Acinetobacter which has a cross reacting epitope with myelin is 4-carboxy-muconolactone-decarboxylase. One molecule present in Acinetobacter which has a cross reacting epitope with neurofilaments is protocatechuate 3,4-dioxygenase.

A test kit for use according to the invention therefore contains at least one test antigen as indicated above or hereinafter.

As indicated in our previous applications, antibodies are assayed and a positive result is indicated by levels of antibodies above that of control samples. Ideally a positive result for any individual sample is indicated when the result is above the 95% or more particularly 99% confidence limits of the control population.

Test protocols in accordance with the present invention are outlined in the following Examples. Test results are illustrated in FIGS. 3 to 23 of the accompanying drawings.

### EXAMPLE 1

Synthesis of Peptides

Peptides may be synthesised by standard solid phase synthesis procedures using Fmoc chemistry. Purification may be achieved using standard HPLC techniques and purity established using mass spectrometry.

ELISA Test A

1) Aliquots of 200 μl/well each containing 5 μg of the diluted suspension of peptide i (having the sequence AIGSRPVDOHLKAL (SEQ ID NO: 11)) or peptide ii (having the sequence QVYYRPVDOYSNQN (SEQ ID NO: 12)) are absorbed onto 96 well flat bottomed rigid polystyrene microtitre plates overnight at 4° C.

2) The plates are then washed 3 times with phosphate buffered saline (PBS), 0.1% (v/v) Tween 20.

3) Aliquots of 200 μl of blocking solution (0.2% w/v ovalbumin, 0.1% v/v Tween 200 in PBS is added to each well and incubated for one hour at 37° C.

4) The plates are then washed 3 times with PBS.Tween 20.

5) Aliquots of 200 μl serum samples (test or control) diluted 1/200 in PBS. Tween 20 is added and incubated for 2 hours at 37° C.

6. The plates are then washed 3 times with PBS.Tween 20.

7) Aliquots of 200 μl of peroxidase conjugated rabbit anti-human Immunoglobulin or rabbit anti-cow Immunoglobulin, diluted 1/4000 (cow) (or 1/1000 for human) with PBS.Tween 20 are added and incubated for 2 hours at 37° C.

8) The plates are then washed 3 times with PBS.Tween 20.

9) The development of the colorimetric assay takes place at room temperature for 20 minutes, after the addition of 200 μl per well of 0.5 mg/ml (2,2′-azinobis(3-ethylbenzthiazoline-6-sulphonic acid) in citrate/phosphate buffer, pH 4.1, containing 0.98 mM hydrogen peroxide.

10) the reaction is then stopped with 100 μl of 2 mg/ml sodium fluoride and optical densities measured at a wavelength of 630 nm with a micro-ELISA plate reader.

### EXAMPLE 2

In order to identify samples containing antibodies capable of binding to antigens present in both Acinetobacter and prions the following ELISA assays (ELISA B and ELISA C) may be performed to look for samples which are positive in both assays. Antibodies are assayed and a positive result is indicated by levels of antibodies above that of control samples. Ideally a positive result for any individual sample is indicated when the result is above the 95% or more particularly 99% confidence limits of the control population.

ELISA Test B

1) Aliquots of 200 μL/well of the diluted suspension of Acinetobacter calcoaceticus (NCIMB 10694, Aberdeen) grown in nutrient broth are absorbed onto 96 well flat bottomed rigid polystyrene microtitre plates overnight at 4° C.

2) The plates are then washed 3 times with phosphate buffered saline (PBS), 0.1% (v/v) Tween 20.

3) Aliquots of 200 lt of blocking solution (0.2% w/v ovalbumin, 0.1% v/v Tween 200 in PBS is added to each well and incubated for one hour at 37° C.

4) The plates are then washed 3 times with PBS.Tween 20.

5) Aliquots of 200 μl serum samples (test or control) diluted 1/200 in PBS. Tween 20 is added and incubated for 2 hours at 37° C.

6. The plates are then washed 3 times with PBS.Tween 20.

7) Aliquots of 200 μl of peroxidase conjugated rabbit anti-human Immunoglobulin or rabbit anti-cow Immunoglobulin, diluted 1/4000 (cow) (or 1/1000 for human) with PBS.Tween 20 are added and incubated for 2 hours at 37° C.

8) The plates are then washed 3 times with PBS.Tween 20.

9) The development of the colorimetric assay takes place at room temperature for 20 minutes, after the addition of 200 μl per well of 0.5 mg/ml (2,2′-azinobis(3-ethylbenzthiazoline-6-sulphonic acid) in citrate/phosphate buffer, pH 4.1, containing 0.98 mM hydrogen peroxide.

10) The reaction is then stopped with 100 μl of 2 mg/ml sodium fluoride and optical densities measured at a wavelength of 630 nm with a micro-ELISA plate reader.

ELISA Test C

1) Aliquots of 200 μl/well each containing 1-10 μg of bovine, human, ovine, or other vertebrate prion are absorbed onto 96 well flat bottomed rigid polystyrene microtitre plates overnight at 4° C.

2) The plates are then washed 3 times with phosphate buffered saline (PBS), 0.1% (v/v) Tween 20.

3) Aliquots of 200 μl of blocking solution (0.2% w/v ovalbumin, 0.1% v/v Tween 200 in PBS is added to each well and incubated for one hour at 37° C.

4) The plates are then washed 3 times with PBS.Tween 20.

5) Aliquots of 200 μl serum samples (test or control) diluted 1/200 in PBS. Tween 20 is added and incubated for 2 hours at 37° C.

6. The plates are then washed 3 times with PBS.Tween 20.

7) Aliquots of 200 μl of peroxidase conjugated rabbit anti-human Immunoglobulin or rabbit anti-cow Immunoglobulin, diluted 1/4000 (cow) (or 1/1000 for human) with PBS.Tween 20 are added and incubated for 2 hours at 37° C.

8) The plates are then washed 3 times with PBS.Tween 20.

9) The development of the colorimetric assay takes place at room temperature for 20 minutes, after the addition of 200 μl per well of 0.5 mg/ml (2,2′-azinobis(3-ethylbenzthiazoline-6-sulphonic acid) in citrate/phosphate buffer, pH 4.1, containing 0.98 mM hydrogen peroxide.

10) The reaction is then stopped with 100 μl of 2 mg/ml sodium fluoride and optical densities measured at a wavelength of 630 nm with a micro-ELISA plate reader.

### EXAMPLE 3

### MAN Index

The new MAN index is a method of combining the measurement of antibodies capable of binding to prions with one or more of:
a) the measurement of antibodies capable of binding to antigens present in myelin, and/or
b) the measurement of antibodies capable of binding to antigens present in neurofilaments, and/or
c) the measurement of antibodies capable of binding to antigens present in Acinetobacter species.

The MAN index is then obtained by multiplying the result from the test to measure antibodies capable of binding to prions (which may be expressed in units of optical density) with the value obtained using the same serum sample when tested for antibodies capable of binding to antigens present in myelin and/or neurofilaments and/or Acinetobacter species. This is performed for both disease positive and control samples. Ideally a positive result for any individual sample is indicated when the result is above the 95% or more particularly 99% confidence limits of the control population.

More usually the MAN index will use the combination of results obtained using ELISA TEST A/ or ELISA with one or more of:
a) antibodies capable of binding to antigens present in Acinetobacter which cross-react with myelin, and
b) antibodies capable of binding to antigens present in Acinetobacter which cross-react with neurofilaments

ELISA TEST A is performed using a peptide containing the sequence RPVDQ (SEQ ID NO: 10), which is usually either of the peptides detailed under ELISA TEST A, alongside ELISA TEST D and/or ELISA TEST E.

ELISA TEST D

An ELISA to test for antibodies capable of binding to myelin.

The methodology is the same as for ELISA TEST A, except that in step 1 the antigen absorbed onto the microtitre plate may be myelin at a concentration of 5 μg/ml (for example bovine myelin from Sigma Chemical Company, Fancy Road, Poole, Dorset, BH12 4XA) or a peptide containing an antigenic component thereof, for example which contains the peptide sequence or RFAWGE (SEQ ID NO: 13) or RFSWGAE (SEQ ID NO: 14) or RFXWXE (SEQ ID NO: 15) or RFXWXXE (SEQ ID NO: 16) (where X is any amino acid), or more ideally QNFISRFAWGEVNSR (SEQ ID NO: 17) or RGSLSRFSWGAEGQK (SEQ ID NO: 18) (at a concentration of 5 μg/ml).

ELISA Test E

An ELISA to test for antibodies capable of binding to neurofilaments.

The methodology is the same as for ELISA TEST A, except that in step 1 the antigen absorbed onto the microtitre plate may be neurofilaments at a concentration of 5 μg/ml (for example bovine neurofilaments from Sigma Chemical Company, Fancy Road, Poole, Dorset, BH12 4XA) or an antigenic component thereof, for example which contains the peptide sequence NEALEK (SEQ ID NO: 3) or LKKVHEE (SEQ ID NO: 4) or EALEKQL (SEQ ID NO: 5) or ELEDKQN (SEQ ID NO: 6) or KKVHEE (SEQ ID NO: 7) or EIRDLR (SEQ ID NO: 8) or EQEIRDLR (SEQ ID NO: 9) or KEALEK (SEQ ID NO: 19) or IEKVEEE (SEQ ID NO: 20) or EALEYGL (SEQ ID NO: 21) or ALEDKSN (SEQ ED NO: 22) or EAYAKQL (SEQ ID NO: 23) or KKVKEE (SEQ ID NO: 24) or EIRDLE (SEQ ID NO: 25) or EQIVRDAR (SEQ ID NO: 26), or more ideally RALIALDKSNFIEA (SEQ ID NO: 27) or KQLQELEDKQNADIS (SEQ ID NO: 28) (at a concentration of 5 μg/ml).

Typical results of tests as described above are shown in the attached FIGS. 3 to 23.

### FIGURE LEGENDS

FIG. 1 shows the 3-dimensional structures of the corresponding parts of the Acinetobacter and prion molecules in which the Aspartic acid and Arginine residues are of especial significance.

FIG. 2 shows the cross-reacting epitope in the prion molecule.

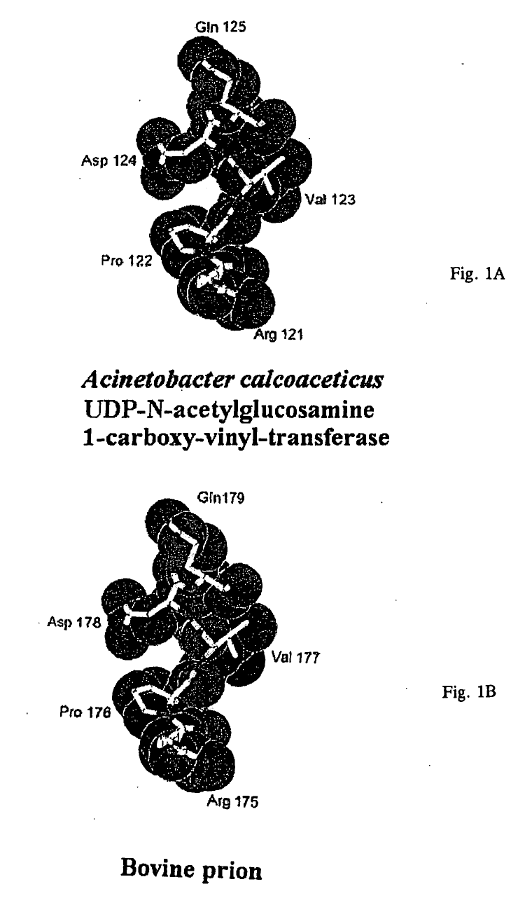

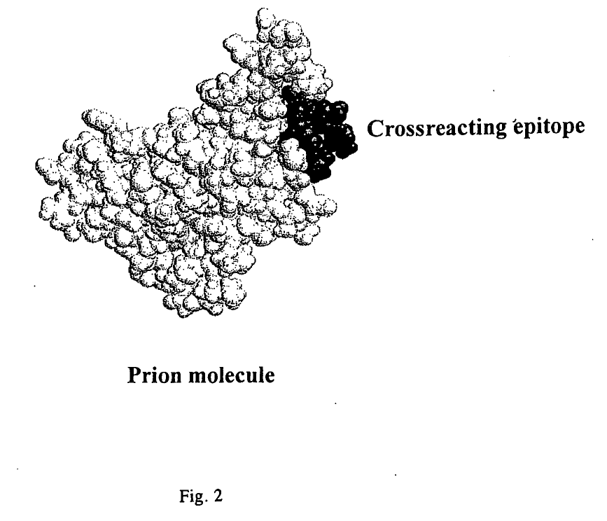

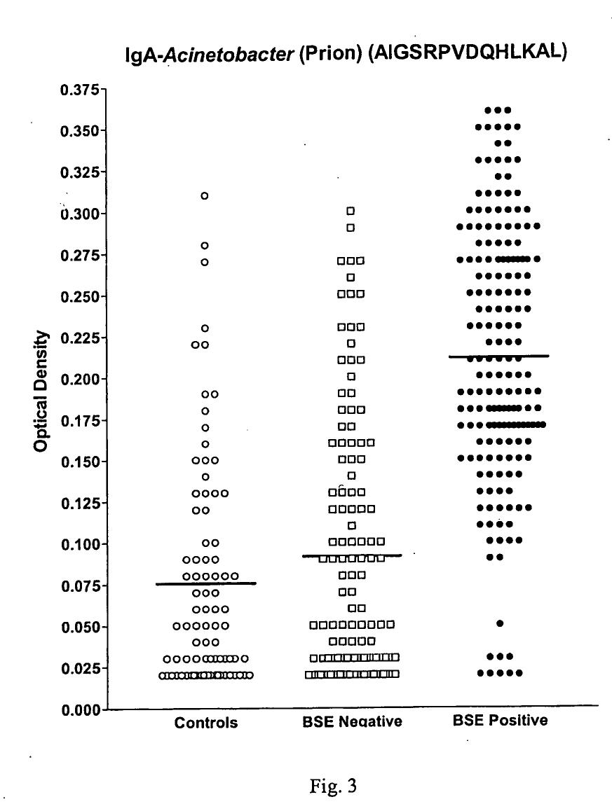

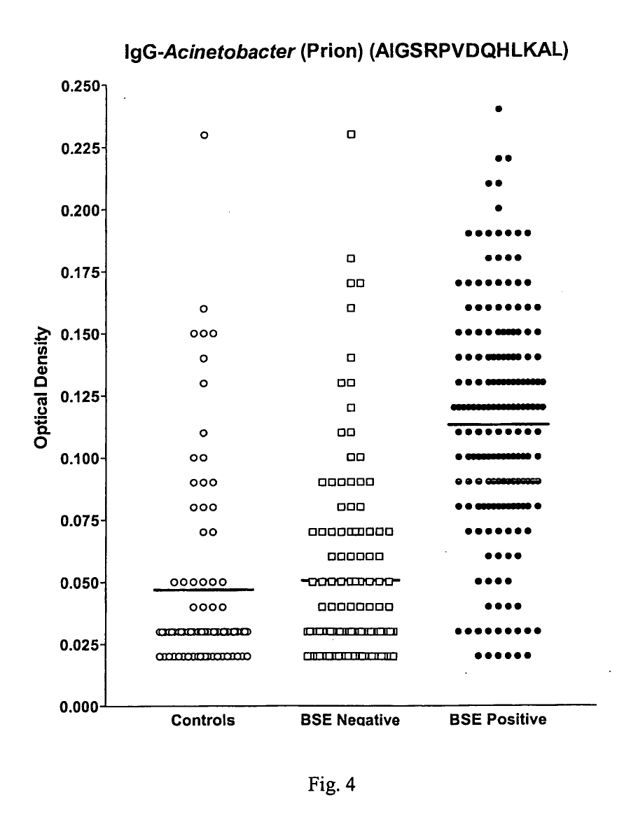

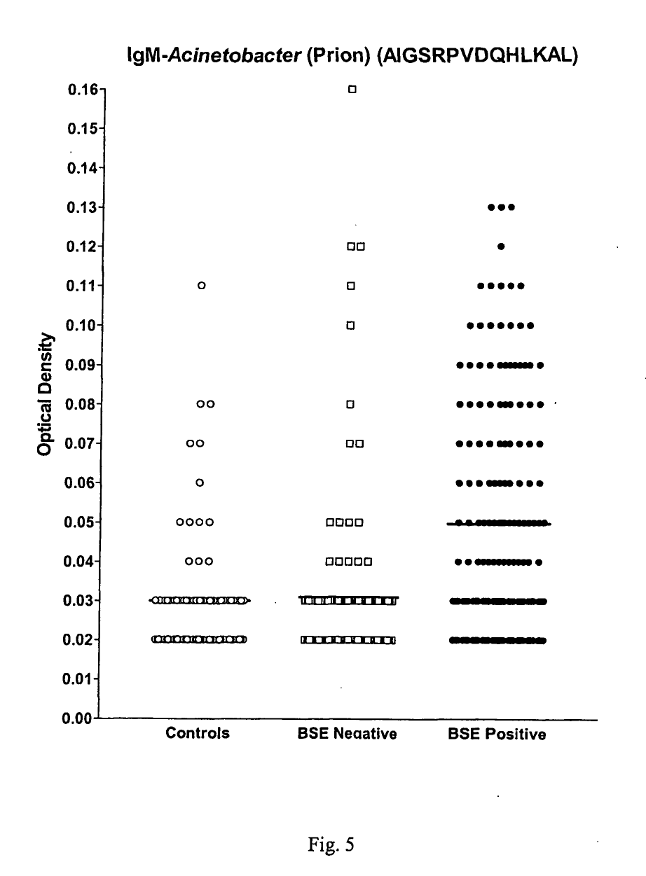

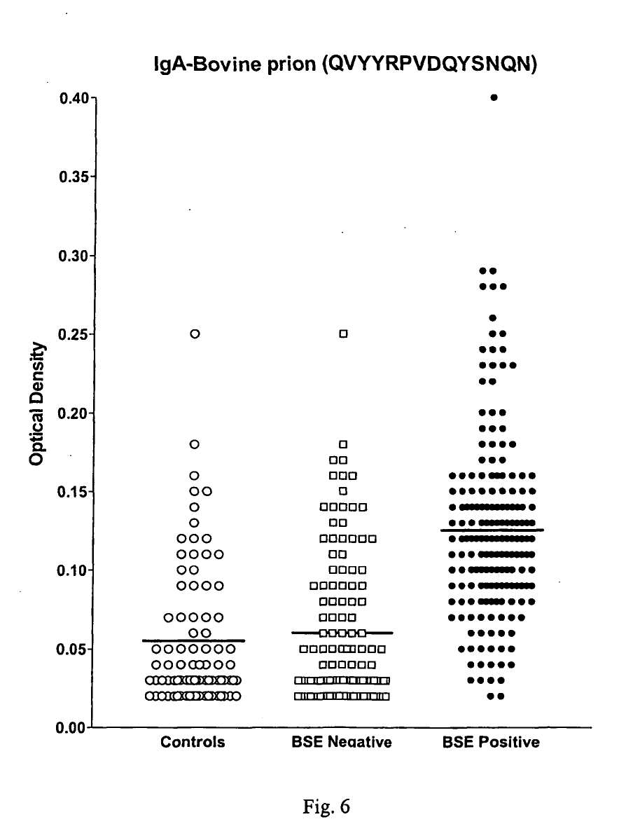

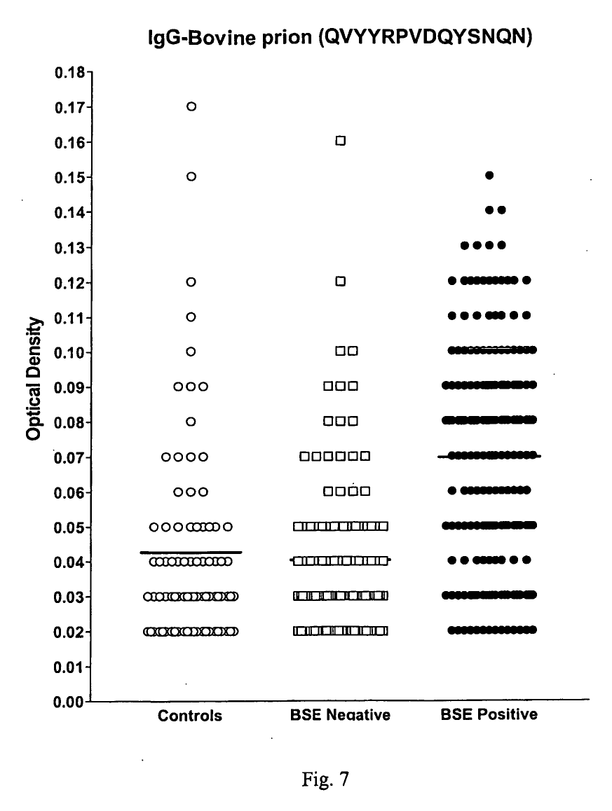

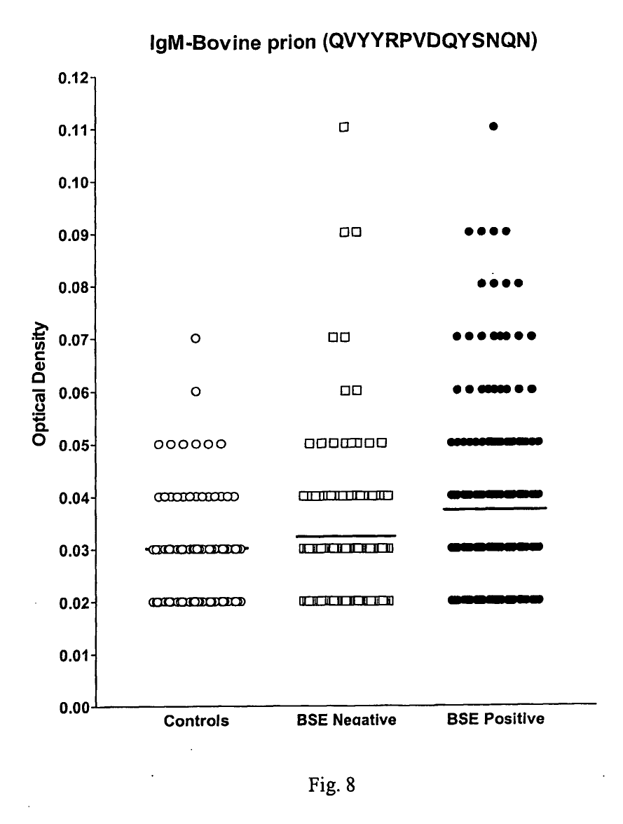

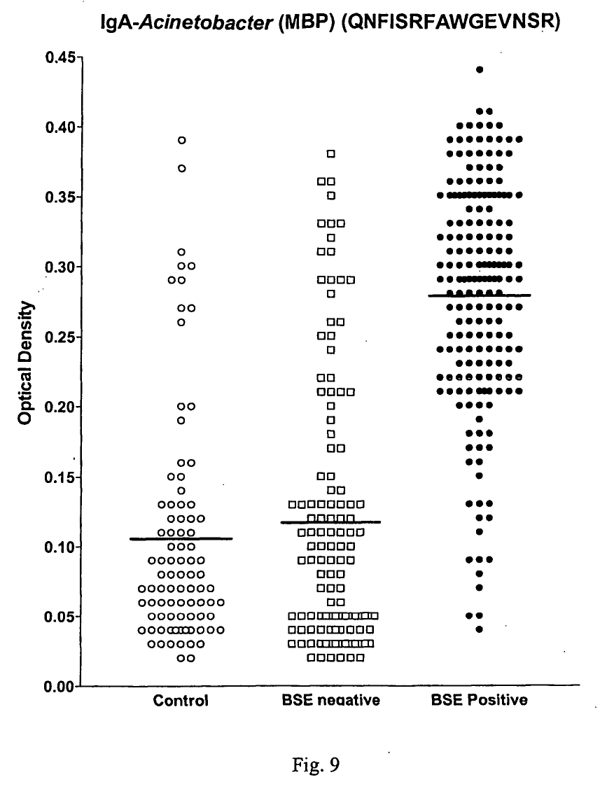

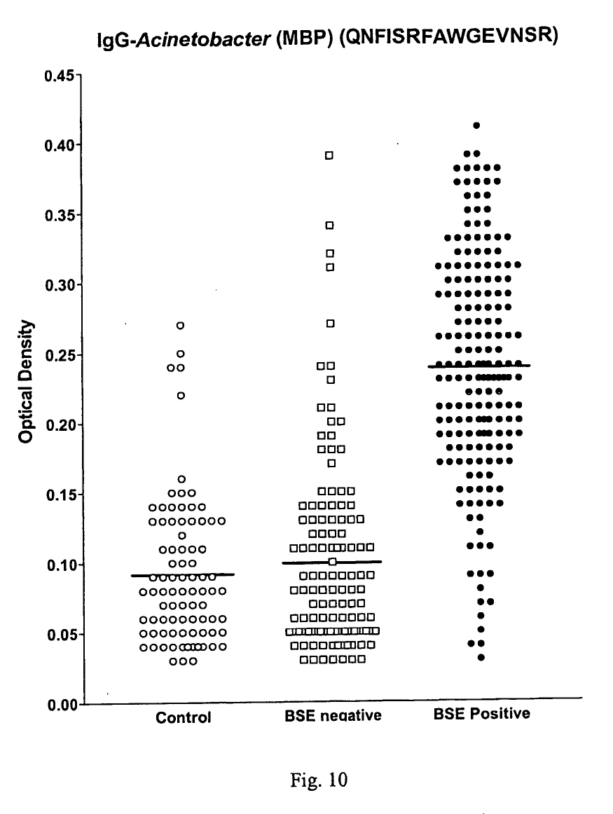

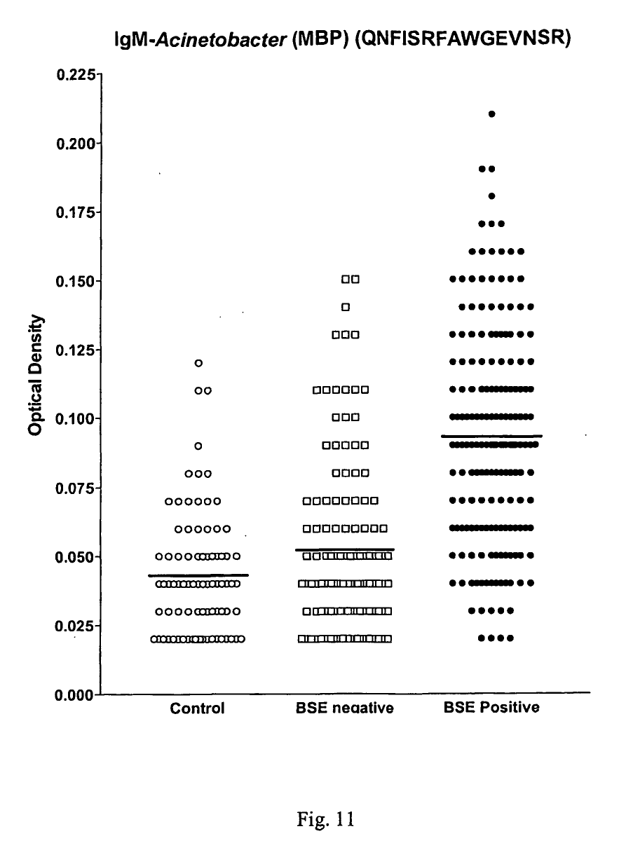

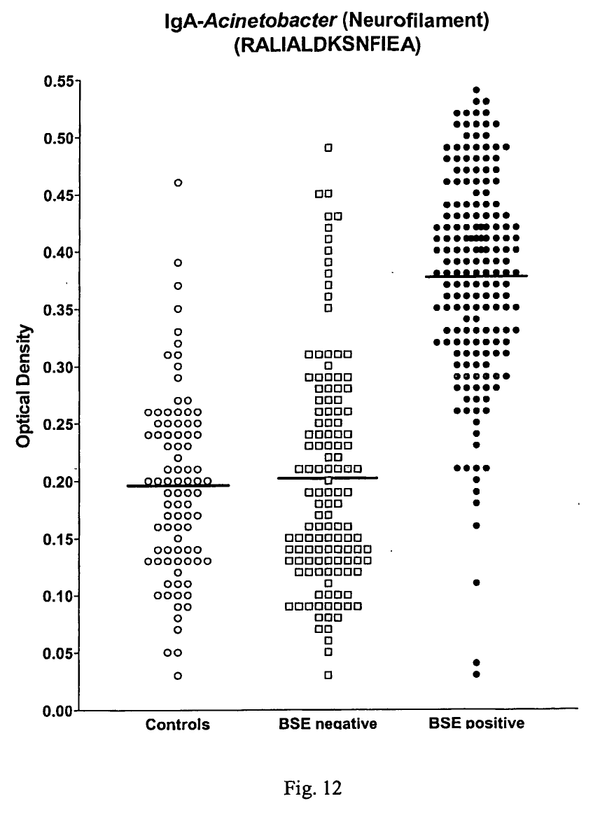

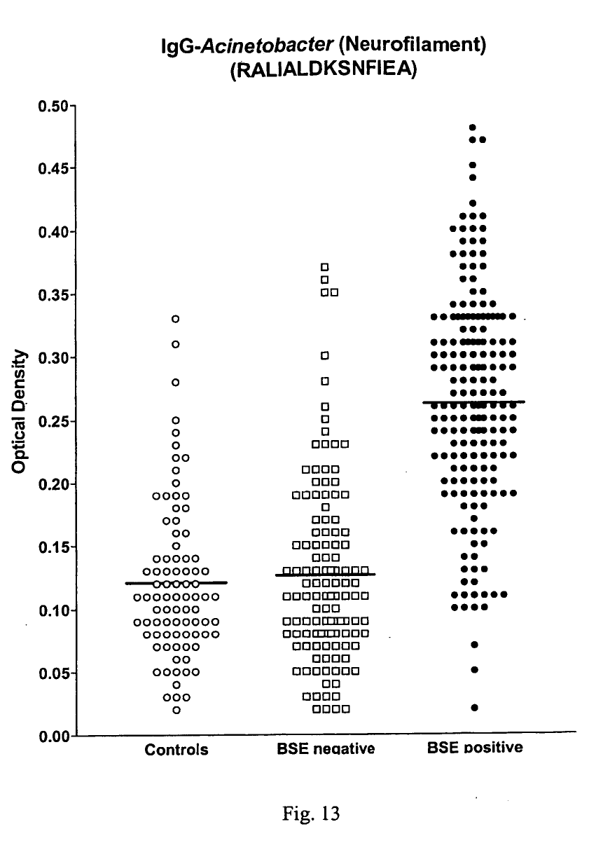

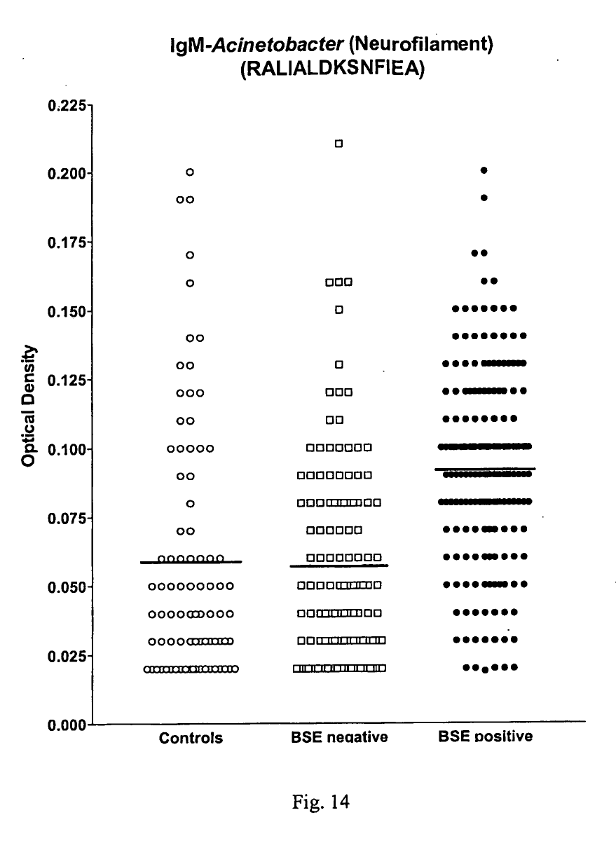

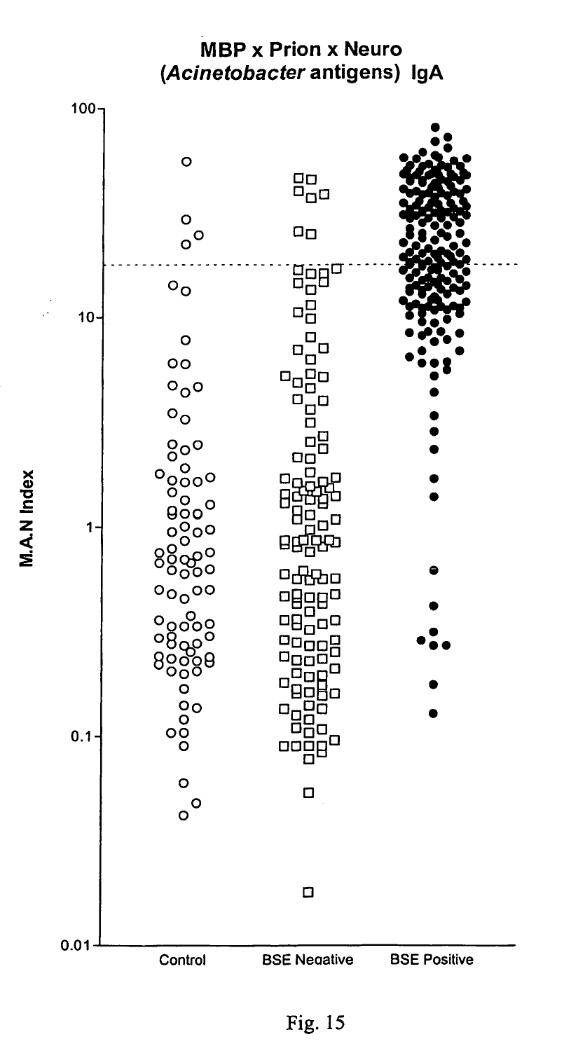

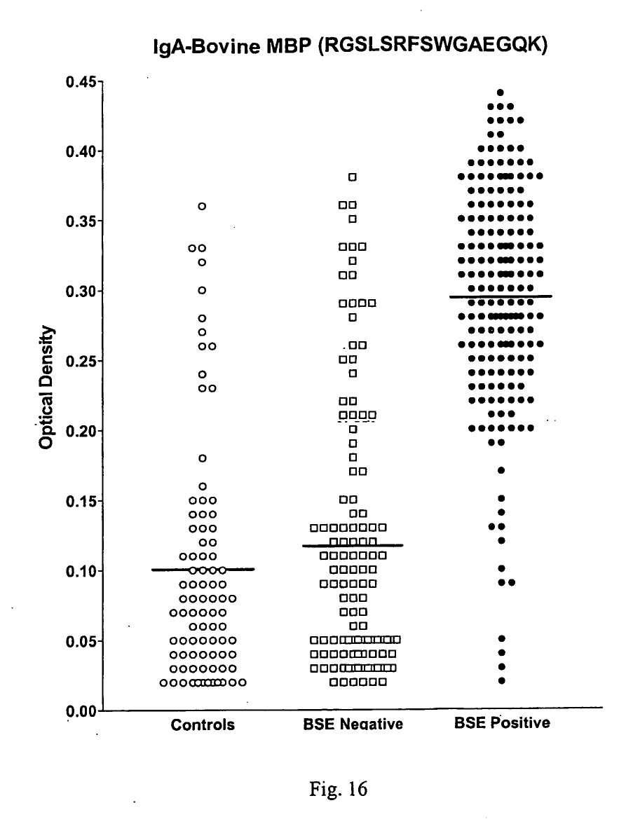

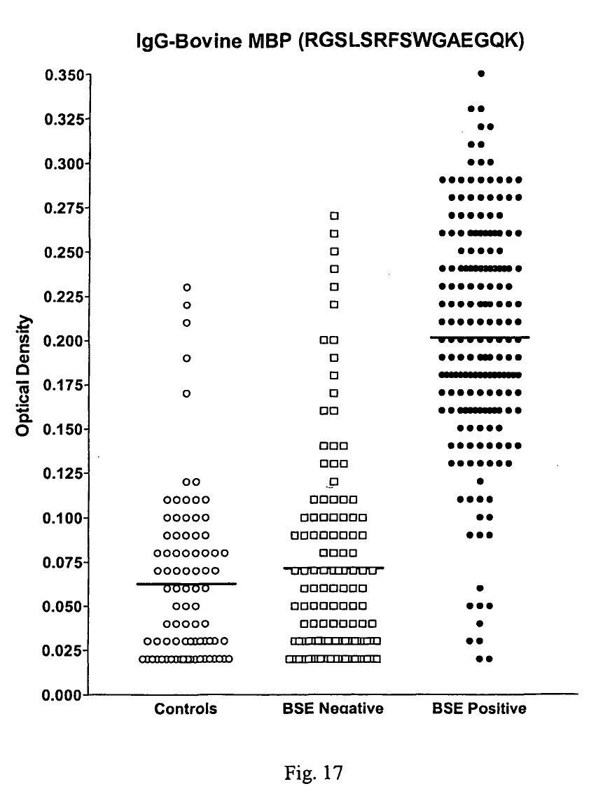

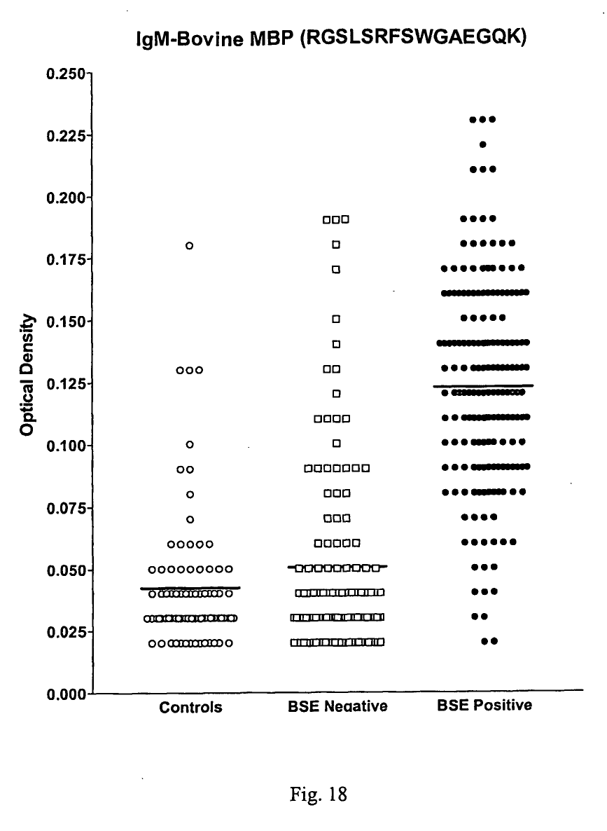

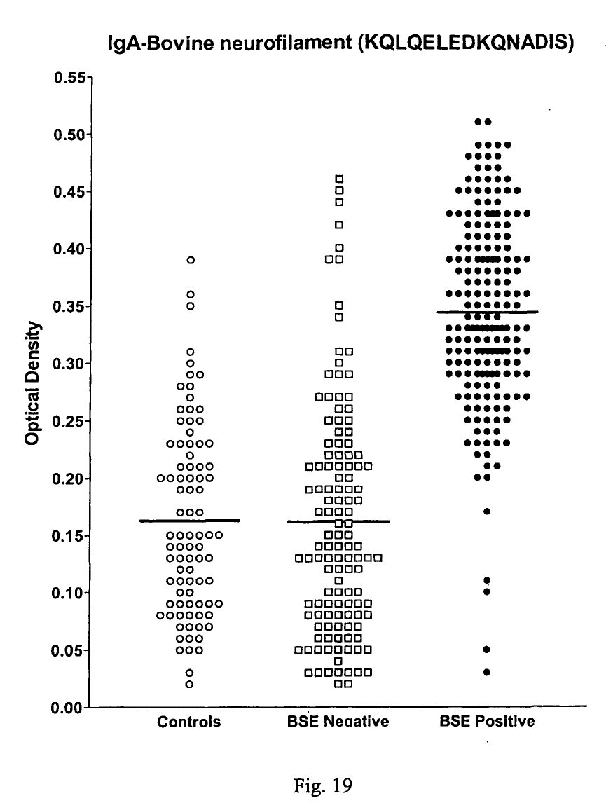

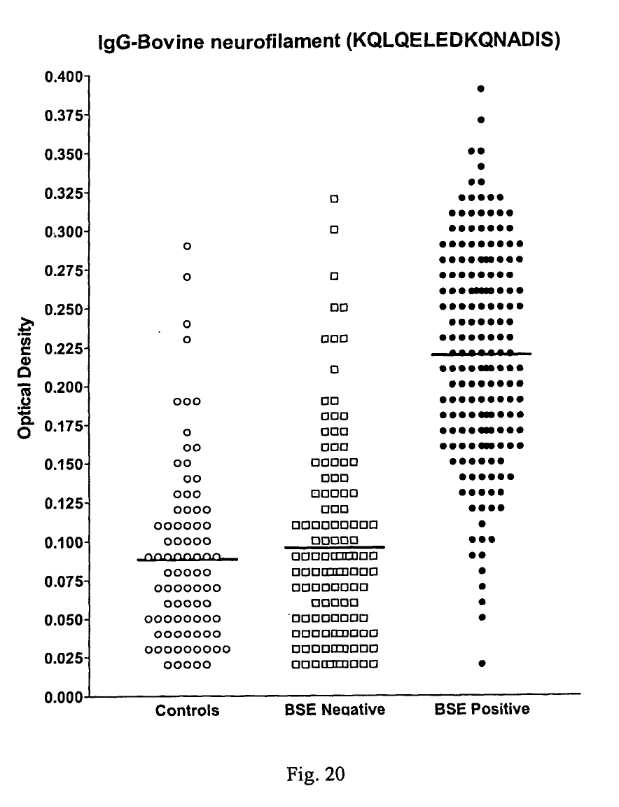

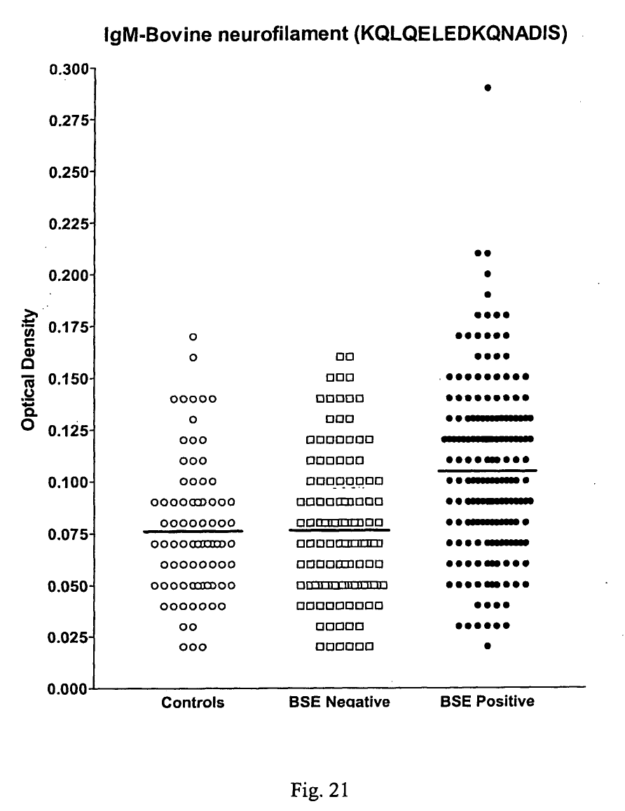

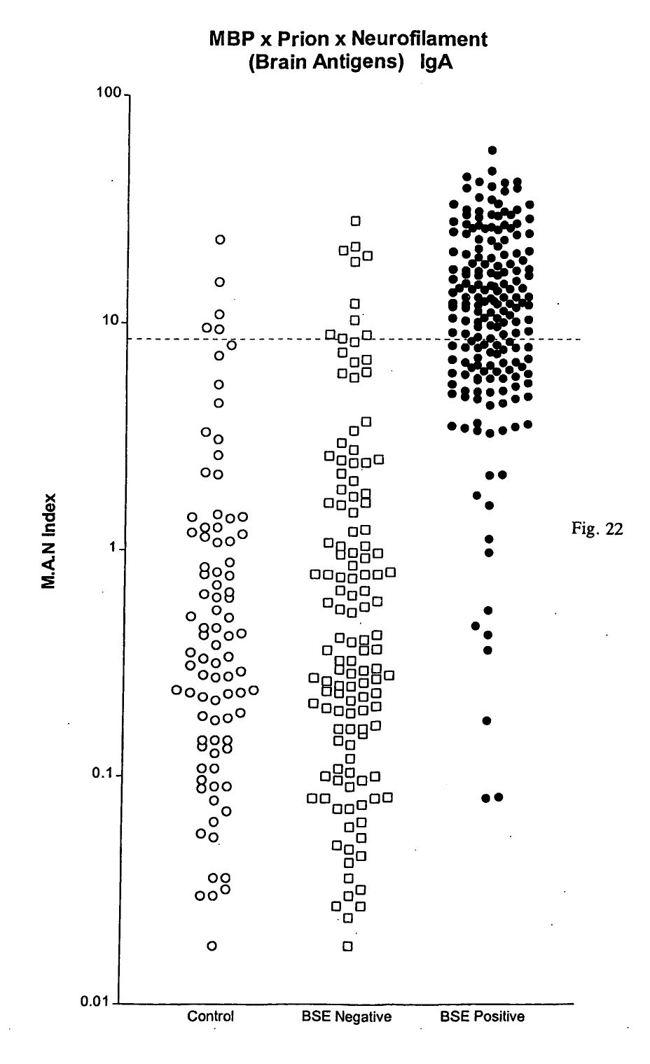

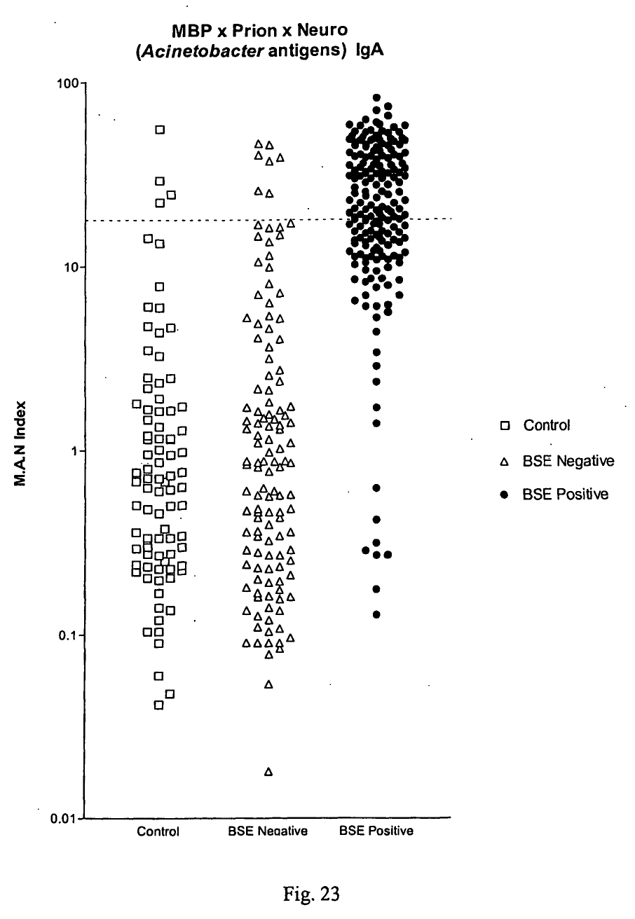

In FIG. 3 through FIG. 23 the following nomenclature is used: Controls are animals which are healthy and have no neurological symptoms, and BSE negative are animals which have been referred to the Central Veterinary Laboratory (CVL) with limping problems and were suspected of having BSE. The animals were sacrificed, brains examined for BSE and no evidence of disease was found by histochemistry, and BSE positive animals have been referred to CVL suspected of having BSE which was confirmed following post mortem and subsequent histological analysis. The horizontal bars on the graphs indicate the mean value for each population.

FIG. 3 shows the results (expressed in optical density units) of the measurement of IgA antibodies to the prion cross-reactive peptide from Acinetobacter in sera of cows with BSE versus normal cows and BSE negative cows tested in ELISA A.

FIG. 4 shows the results (expressed in optical density units) of the measurement of IgG antibodies to the prion cross-reactive peptide from Acinetobacter in sera of cows with BSE versus normal cows and BSE negative cows tested in ELISA A.

FIG. 5 shows the results (expressed in optical density units) of the measurement of IgM antibodies to the prion cross-reactive peptide from Acinetobacter in sera of cows with BSE versus normal cows and BSE negative cows tested in ELISA A.

FIG. 6 shows the results (expressed in optical density units) of the measurement of IgA antibodies to the Acinetobacter cross-reactive peptide from bovine prions in sera of cows with BSE versus normal cows and BSE negative cows tested in ELISA A.

FIG. 7 shows the results (expressed in optical density units) of the measurement of IgG antibodies to the Acinetobacter cross-reactive peptide from bovine prions in sera of cows with BSE versus normal cows and BSE negative cows tested in ELISA A.

FIG. 8 shows the results (expressed in optical density units) of the measurement of IgM antibodies to the Acinetobacter cross-reactive peptide from bovine prions in sera of cows with BSE versus normal cows and BSE negative cows tested in ELISA A.

FIG. 9 shows the result (expressed in optical density units) of the measurement of IgA antibodies to the myelin cross-reactive peptide from Acinetobacter in sera of cows with BSE versus normal cows and BSE negative cows tested in ELISA D.

FIG. 10 shows the result (expressed in optical density units) of the measurement of IgG antibodies to the myelin cross-reactive peptide from Acinetobacter in sera of cows with BSE versus normal cows and BSE negative cows tested in ELISA D.

FIG. 11 shows the result (expressed in optical density units) of the measurement of IgM antibodies to the myelin cross-reactive peptide from Acinetobacter in sera of cows with BSE versus normal cows and BSE negative cows tested in ELISA D.

FIG. 12 shows the result (expressed in optical density units) of the measurement of IgA antibodies to the neurofilament cross-reactive peptide from Acinetobacter in sera of cows with BSE versus normal cows and BSE negative cows tested in ELISA E.

FIG. 13 shows the result (expressed in optical density units) of the measurement of IgG antibodies to the neurofilament cross-reactive peptide from Acinetobacter in sera of cows with BSE versus normal cows and BSE negative cows tested in ELISA E.

FIG. 14 shows the result (expressed in optical density units) of the measurement of IgM antibodies to the neurofilament cross-reactive peptide from Acinetobacter from sera of cows with BSE versus normal cows and BSE negative cows tested in ELISA E.

FIG. 15 shows the result for each sera of the multiplication of the results (expressed in optical density units) and obtained by measuring IgA antibodies to Acineto antigens which mimic myelin basic protein, prions, and neurofilaments and obtained in FIGS. 3, 9 and 12 according to the new MAN index.

FIG. 16 shows the result (expressed in optical density units) of the measurement of IgA antibodies to the Acinetobacter cross-reactive peptide from bovine myelin from sera of cows with BSE versus normal cows and BSE negative cows tested in ELISA D.

FIG. 17 shows the result (expressed in optical density units) of the measurement of IgG antibodies to the Acinetobacter cross-reactive peptide from bovine myelin in sera of cows with BSE versus normal cows and BSE negative cows tested in ELISA D.

FIG. 18 shows the result (expressed in optical density units) of the measurement of IgM antibodies to the Acinetobacter cross-reactive peptide from bovine myelin in sera of cows with BSE versus normal cows and BSE negative cows tested in ELISA D.

FIG. 19 shows the result (expressed in optical density units) of the measurement of IgA antibodies to the Acinetobacter cross-reactive peptide from neurofilaments in sera of cows with BSE versus normal cows and BSE negative cows tested in ELISA E.

FIG. 20 shows the result (expressed in optical density units) of the measurement of IgG antibodies to the Acinetobacter cross-reactive peptide from neurofilaments in sera of cows with BSE versus normal cows and BSE negative cows tested in ELISA E.

FIG. 21 shows the result (expressed in optical density units) of the measurement of IgM antibodies to the Acinetobacter cross-reactive peptide from neurofilaments in sera of cows with BSE versus normal cows and BSE negative cows tested in ELISA E.

FIG. 22 shows the results for each sera of the multiplication of the results (expressed in optical density units) obtained by measuring IgA antibodies to myelin basic protein, prion, and neurofilaments which mimic Acinetobacter antigens, and obtained in FIGS. 6, 16, and 19 according to the new MAN index.

FIG. 23 shows the results for each sera of the multiplication of the results (expressed in optical density units) obtained by measuring IgA antibodies to Acinetobacter antigens which mimic myelin basic protein, prion, and neurofilaments.

1. A method for detecting a de-myelinating disease or spongiform encephalopathy in vertebrates, including BSE, MS and CJD, which comprises testing a biological sample obtained from the vertebrate for antibodies capable of binding to an antigen present in a prion of vertebrate origin or to one or more antigenic parts (epitopes) thereof.

2. A method according to claim 1, in which the antibodies are also capable of binding to antigens present in Acinetobacter or a part thereof.

3. A method according to claim 1, for detecting a de-myelinating disease or spongiform encephalopathy in vertebrates which comprises testing for antibodies capable of binding to an epitope which contains the peptide sequence RPVDQ (SEQ ID NO: 10) or a related sequence.

4. A method according to claim 3, in which the epitope has or contains the sequence AIGSRPVDQHLKAL (SEQ ID NO: 11) or QVYYRPVDQYSNQN (SEQ ID NO: 12).

5. A method according to any one of claims 1 to 4, in which the antibodies are IgA antibodies.

6. A method according to any one of claims 1 to 4, in which the antibodies are IgG antibodies.

7. A method according to any one of claims 1 to 4, in which the antibodies are IgM antibodies.

8. A method according to any one of claims 1 to 4 in which a positive result is indicated by levels of antibodies at least about two standard deviations above that of control samples.

9. A method according to any one of claims 1 to 4 combined with an assay for antibodies to myelin or a part thereof.

10. A method according to any one of claims 1 to 4 combined with an assay for antibodies to neurofilaments or a part thereof.

11. A method according to any one of claims 1 to 4 combined with an assay for antibodies to Acinetobacter species or a part thereof.

12. A method for detecting a de-myelinating disease or spongiform encephalopathy in vertebrates, including BSE, MS and CJD, which comprises testing a biological sample obtained from the vertebrate to measure antibodies capable of binding to an antigen present in a prion of vertebrate origin or to one or more antigenic parts (epitopes) thereof, and testing a biological sample or samples from the same vertebrate to measure antibodies to one or more of vertebrate myelin, vertebrate neurofilaments, Acinetobacter species, and antigenic parts of these, and combining the two or more such measurements taken.

13. A method for detecting a demyelinating disease or spongiform encephalopathy in a vertebrate, including BSE, MS, and CJD, which comprises testing a biological sample taken from the vertebrate for
(a) antibodies that bind to a test antigen comprising a prion molecule or a peptide having a sequence present therein, and/or
(b) antibodies that bind to a test antigen comprising an Acinetobacter species or a substance having the sequence or structure of a component of said species,
said prion molecule and said Acinetobacter species having component sequences which mimic vertebrate myelin, and said antigens being sufficiently alike to be cross-reactive with each other and which may themselves also mimic the myelin of the vertebrate, and combining the measurement of said antibodies with corresponding measurements obtained by testing the sample for one or both of the following:
(i) antibodies to the corresponding myelin basic protein or an antigenic component thereof, and
(ii) antibodies to the corresponding neurofilaments or an antigenic component thereof,
said combination of measurements being indicative of the presence or absence of the disease being tested for.

14. A method for detecting a demyelinating disease or spongiform encephalopathy in a vertebrate, including BSE, MS, and CJD, which comprises testing a biological sample taken from the vertebrate for
(a) antibodies that bind to a test antigen comprising a prion molecule or a peptide having a sequence present therein, and/or
(b) antibodies that bind to a test antigen comprising an Acinetobacter species or a substance having the sequence or structure of a component of said species,
said prion molecule having a peptide which mimics antigens in Acinetobacter species and said myelin as well as neurofilament molecules which mimic antigens in Acinetobacter species, said antigens being sufficiently alike that antibodies produced against them in the vertebrate will be cross-reactive, and combining the measurements of said antibodies with corresponding measurements obtained by testing the sample for one, two, or three of the following:
(i) antibodies to the corresponding myelin basic protein or peptide component thereof, or a mimicking antigen in Acinetobacter species, and
(ii) antibodies to the corresponding neurofilaments or a peptide component thereof, or a mimicking antigen in Acinetobacter species, and
(iii) antibodies to the corresponding prion or peptide thereof or a mimicking antigen in Acinetobacter species,
said combination of measurements being indicative of the presence or absence of the disease being tested for.

15. A method according to claim 13 or 14, in which the antigen present in the prion has a sequence specified in the foregoing description.

16. A method according to claim 13 or 14, 14 or 15, in which the antigen present in the Acinetobacter species has a sequence specified in the foregoing description.

17. A method according to claim 13 or 14, in which the measurements are combined by multiplication.

18. A test kit for detecting a demyelinating disease or spongiform encephalopathy in a vertebrate, including BSE, MS, and CJD, the kit comprising an antigen, epitope, or epitopes as test antigen or antigens, the antigen, epitope, or epitopes being disposed in a suitable container, and further wherein the antigen, epitope, or epitopes are selected from the group consisting of a prion molecule or a peptide having a sequence present therein, and an Acinetobacter species or a substance having the sequence or structure of a component of said species.

19. A test kit according to claim 18, in which the test antigens are selected from the group consisting of RFSAWGAE (SEQ ID NO: 1), ISRFAWGEV (SEQ ID NO: 2), NEALEK (SEQ ID NO: 3), LKKVHEE (SEQ ID NO: 4), EALEKQL (SEQ ID NO: 5), ELEDKQN (SEQ ID NO: 6), KKVHEE (SEQ ID NO: 7), EIRDLR (SEQ ID NO: 8), EQEIRDLR (SEQ ID NO: 9), KEALEK (SEQ ID NO: 19), IEKVEEE (SEQ ID NO: 20), EALEYGL (SEQ ID NO: 21), ALEDKSN (SEQ ID NO: 22), EAYAKQL (SEQ ID NO: 23), KKVKEE (SEQ ID NO: 24), EIHMLE (SEQ ID NO: 25), EQIVRDAR (SEQ ID NO: 26), RALIALDKSNFIEA (SEQ ID NO: 27), KQLQELEDKQNADIS (SEQ ID NO: 28), RPVDQ (SEQ ID NO: 10), and sequences containing up to 15 amino acid residues which include RPVDQ (SEQ ID NO: 10), such as AIGSRPVDQHLKAL (SEQ ID NO: 11), and QVYYRPVDQYSNQN (SEQ ID NO: 12).

20. A test kit according to claim 18, comprising an ELISA test kit.
